(19) United States
(12) Patent Application Publication
(10) Pub. No.: US 2013/0164703 A1
(43) Pub. Date: Jun. 27, 2013

(54) SYSTEM, METHOD AND APPARATUS FORREDUCTION OF PAIN AT AN INJECTION SITE

(71) Applicant: David A. Beach, Lutz, FL (US)

(72) Inventor: David A. Beach, Lutz, FL (US)

(21) Appl. No.: 13/335,987

(22) Filed: Dec. 23, 2011

(51) Int. Cl. A61M 5/42; A61C 19/08; A61B 1/247

(52) U.S. Cl. 433/31; 433/82; 433/215

(57) ABSTRACT

A device for reducing pain having an elongated shaft for grasping with a hand. The elongated shaft has a blunt end member. The blunt end member is shaped to allow a practitioner to apply force from the blunt end member onto a target tissue before using a syringe and needle to inject a fluid (e.g. anesthesia) into the tissue. There is a bore (hole) in the blunt end member, through which the needle is passed and subsequently, pushed into the tissue for injection of the fluid.

### FIELD

The present invention relates to an apparatus for performing medical and dental injections, and in particular to such a method and apparatus for performing such injections while reducing the pain associated with the injection.

### BACKGROUND

Often, to reduce pain of a medical procedure, one must endure the pain of an injection of an anesthetic. Depending upon where the injection is made, the actual pain of the injection ranges from mild pain to high levels of pain. For example, a high level of pain occurs when injections are made in the palate, due to the sensitivity of the palate. These types of injections are often made into the palate for various types of dental procedures. Because of high levels of pain associated with medical/dental procedures, many medical/dental patients either delay care or avoid treatment altogether. There are many known consequences of delaying or avoiding, for example, dental procedures, including worsened tooth decay, tooth loss, abscesses, and in some extreme cases, death.

Patients who wish to avoid the pain associated with palate injections for these types of procedures must either forego anesthesia, or resort to full or partial sedation. The former is often too painful, especially for those who are adverse to the pain of the injection in the first place. The later is expensive, time consuming, and has increased patient risks associated with general anesthesia. Sedation dentistry also requires an additional person who will take the patient home while the patient recovers from the anesthesia. These alternatives are not very good solutions for most patients and thus, palatal injections remain the most common way to provide anesthesia for many dental procedures.

Some practitioners apply a topical anesthesia before the injection is made. The topical anesthesia only partially reduces the pain to the patient. During the injection, the needle reaches further into the tissue than reached by the topical anesthetic and is, therefore, ineffective.

Some practitioners stimulate the patient's cheek or jaw to distract the patient from focusing on the needle penetrating the palate. Some practitioners use a cryogenic spray to rapidly cool the skin surface before the injection is made. All have limited success.

“Pain Gate” theory is based on studies showing that small-diameter nerve pain fibers and large-diameter touch/pressure/vibration fibers share “gates” within the body. Large-diameter nerve fibers passing through the same gate as the small-diameter fibers are capable of inhibiting the pain stimuli from the smaller nerves. Pain Gate studies also found that chemicals released as a response to the pain stimuli influence whether the gate is open or closed. These studies show that stimulating the periphery of the pain site interferes with pain stimuli from the smaller nerves.

One way to shut the pain gate is by stimulating those nerves responsible for carrying the touch signal. For example, if one stimulates the nerves in the area of a source of pain by rubbing or exerting pressure, the sense of pain perceived by the brain is reduced.

The art includes a number of devices that are intended to reduce a patient's perceived pain resulting from an injection or other medical procedure, most of which provide vibration energy in the area where the injection is to be made. For example, U.S. Pat. Pub. No.2008/0086159 to Zweifler has a guide that provides vibrational energy around the site of an injection to reduce pain. U.S. Pat. No. 6,231,531 to Lum et al. has a blood sample device that surrounds the skin-piercing point with a vibrating surface. U.S. Pat. No. 3,620,209 to Kravitz has a device to be used in conjunction with a traditional syringe injection for reducing the sensation of pain felt by the patient. The device comprises a horseshoe-shaped vibrational element that is strapped to the patient's body, roughly circumscribing the area where the injection will be made.

Devices that provide vibrational energy to the area in which an injection will be made have been shown to reduce pain. Providing such vibrational energy is difficult, especially in areas of limited access such as a patient's mouth, e.g. the palate.

Some practitioners try to apply pressure in an area around where the injection will be made using, for example, a dental mirror. This has shown to help reduce pain, but it is difficult to position such a device. Additionally, with such a device pressure can only be exerted in a limited area at one side of the injection target. It is also too easy for such a device to slip while the injection is being made.

What is needed is a device that will apply pressure around an area in which an injection will be made, thereby closing the Pain Gate and reducing the sensation of pain from an injection.

### SUMMARY

A device for reducing pain has an elongated shaft for grasping with a hand and a blunt end member. The blunt end member is shaped to allow a practitioner to apply force from the blunt end member onto a target tissue before using a syringe and needle to inject a fluid (e.g. anesthesia) into the tissue. There is a bore (hole) in the blunt end member, through which the needle is passed and subsequently, pushed into the tissue for injection of the fluid.

In one embodiment, a device for reducing pain is disclosed including an elongated shaft having a first end and a distal second end with a blunt end member at the first end. At least one bore passing through the blunt end member. The at least one bore has an inner diameter greater than an outer diameter of a needle.

In another embodiment, a method of reducing pain from an injection is disclosed including holding an elongated shaft having a first end and a distal second end and pressing a blunt end member against a tissue of a patient. The blunt end member is at the first end of the elongated shaft. Next, a needle of a syringe is passed through a bore and into the tissue. The bore in cut or formed in the blunt end member and has an inner diameter greater than an outer diameter of the needle. Next, a fluid from the syringe is injected into the tissue by pressing a plunger of the syringe, then the needle is removed from the tissue and from the bore, and pressure is released from the blunt end member on the tissue.

In another embodiment, a device for reducing pain is disclosed including, an elongated shaft that has a first end and a distal second end. At the first end of the elongated shaft is a blunt end member. The blunt end member has a rounded surface facing away from the elongated shaft with a bore passing through the blunt end member and the rounded surface. The bore has a diameter greater than a diameter of a needle.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a schematic view of a device for reducing pain being used in a patient's mouth.

FIG. 2 illustrates a cross sectional view of the device for reducing pain being used with a hypodermic needle.

FIG. 3 illustrates a top plan view of the device for reducing pain.

FIG. 4 illustrates a cross sectional view of the device for reducing pain.

FIG. 5 illustrates a cross sectional view of the device for reducing pain with optional end mirror.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

Throughout this description, a particular shape/style of device is shown for exemplary reasons. Any of a variety of shapes, sizes, materials, and dimensions are fully anticipated.

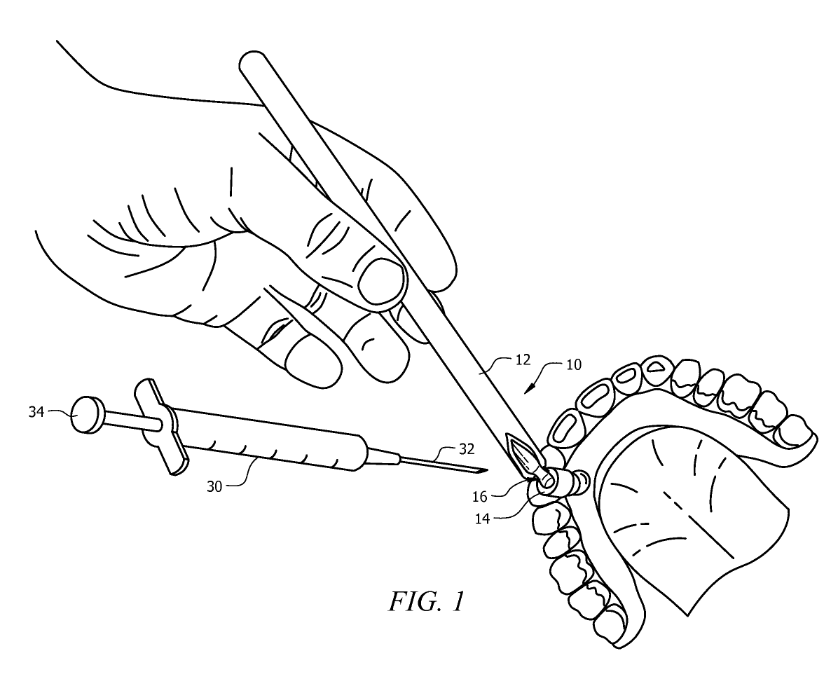

Referring to FIG. 1, a schematic view of a device for reducing pain 10 being used in a patient's mouth is shown. In this view, the device for reducing pain 10 is pressed up against a patient's gums. The device for reducing pain 10 has an elongated shaft 12, shown being held in a practitioner's hand (e.g. dentist, dental assistant). The blunt end member 14 of the device for reducing pain 10 is in contact with the patient's gums and is pressed, thereby shutting the pain gate through the use of pressure stimulus. Pressure is exerted while the needle 32 of a syringe 30 is passed through a bore 16 in the blunt end member 14 of the device for reducing pain 10. The bore 16 is preferably large enough to allow passage of at least one gauge of needle 32. For example, the inner diameter of the bore 16 is greater than an outer diameter of needle 32. The bore 16 is typically sized large enough for all gauges of needles 32 used by a particular practitioner (e.g. dentist, doctor).

After the needle is passed through the bore 16 and into the gums of the patient, the plunger 34 is pressed to deliver the anesthesia to the gums of the patient.

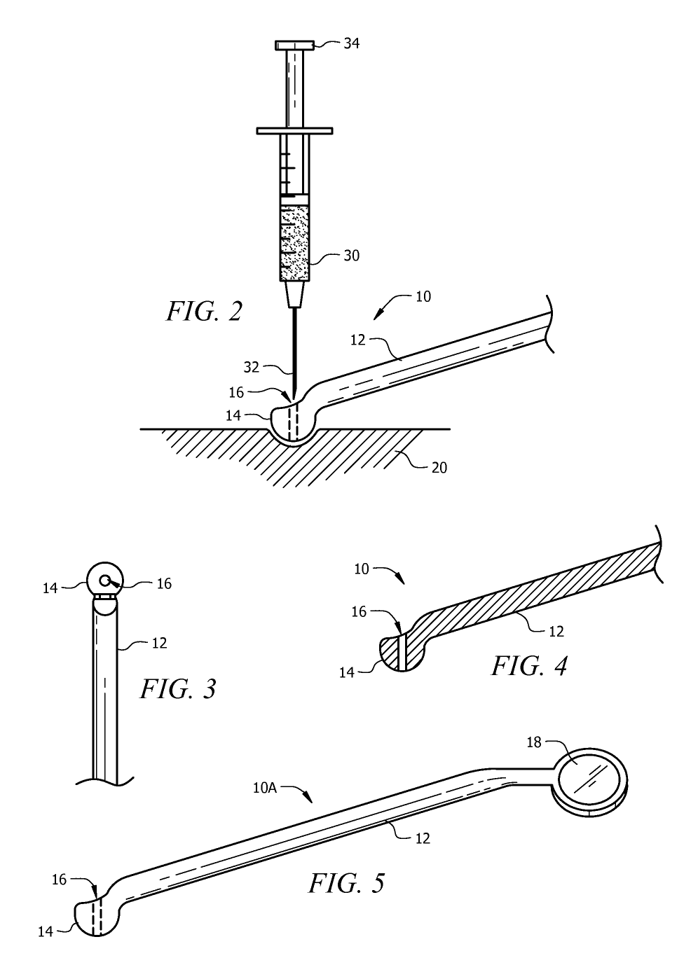

Referring to FIG. 2, a cross sectional view of the device for reducing pain 10 being used with a hypodermic needle is shown. In this view, the device for reducing pain 10 is pressed up against a patient's gums 20 (note that the patient's gums 20 are slightly indented). Pressure is provided to the device for reducing pain 10 along the elongated shaft 12. The blunt end member 14 of the device for reducing pain 10 is in contact with, and slightly displacing, the patient's gums. The pressure acts to shut the pain gate. Note that while the contact surface of the blunt end member 14 is shown as rounded to improve patient comfort, this is not a requirement. Any shape of blunt end member 14 is anticipated.

The bore 16 is preferably large enough to allow passage of at least one gauge of needle 32. The bore 16 is typically sized large enough for all gauges of needles 32 used by a particular practitioner (e.g. dentist, doctor).The needle 32 of a syringe 30 is shown in position to be passed through a bore 16 in the blunt end member 14 of the device for reducing pain 10 and, eventually, into the gums 20 of the patient.

Referring to FIG. 3, a top plan view of the device for reducing pain 10 is shown in which the bore 16 is visible. Note that the bore 16 is shown as a circular (tubular) bore 16, though any shape of a bore 16 is anticipated (e.g. oval, square).

Referring to FIG. 4, a cross sectional view of the device for reducing pain 10 is shown, exposing the bore 16 in the blunt end member 14.

Referring to FIG. 5, a cross sectional view of the device for reducing pain 10A with optional end mirror 18 is shown. Although the device for reducing pain 10 is anticipated to have any possible end style or shape such as shown in FIG. 1, it is fully anticipated that other end styles are incorporated. Examples of alternative end styles or dental devices include the mirror end 18 (as in FIG. 5) as well as any other end style, including, but not limited to, a dental pick, scraper, etc.

Although the elongated shaft 12 is shown in a simple tubular shape, any size and shape of the elongated shaft 12 is anticipated, including, but not limited to, elongated shafts 12 with gripping sections, elongated shafts 12 with texture for better gripping, curved elongated shafts 12, etc.

It is anticipated that the device for reducing pain 10 is made of any relatively stiff material, including, but not limited to, metal, hard plastic, hard rubber, etc. It is also anticipated that the device for reducing pain 10 is made of a combination of materials. For example, a stiff metal inner portion and a soft rubber outer coating. It is anticipated that some materials will provide a limited amount of flexibility as long as sufficient pressure is provided to trigger the pain gate.

In some devices for reducing pain 10, the blunt end member 14 is coated with a slip-reducing material to improve comfort and/or reduce slippage (e.g. across wet mouth tissue). An example of such a material is rubber.

Although a single bore 16 through the blunt end member 14 is shown, any number of bores 16 is anticipated. For example, in a practice in which there are two types of syringes used, each having a different gauge, the blunt end member 14, for example, has two bores 16, one for each needle gauge.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A device for reducing pain, the device comprising:
an elongated shaft having a first end and a distal second end; and
a blunt end member at the first end of the elongated shaft, the blunt end member having at least one bore through the blunt end member, the at least one bore having an inner diameter greater than an outer diameter of a needle.

2. The device for reducing pain of claim 1, wherein the elongated shaft is linear.

3. The device for reducing pain of claim 1, wherein the blunt end member has a curved surface for pressing against a tissue of a patient.

4. The device for reducing pain of claim 3, wherein the curved surface is coated with a slip-reducing material.

5. The device for reducing pain of claim 4, wherein the slip-reducing material is rubber.

6. The device for reducing pain of claim 1, further comprising a dental device affixed at the distal second end of the elongated shaft.

7. The device for reducing pain of claim 6, wherein the dental device is a dental mirror.

8. The device for reducing pain of claim 6, wherein the dental device is a dental pick.

9. The device for reducing pain of claim 6, wherein the dental device is a dental scraper.

10. A method of reducing pain from an injection, the method comprising:
holding an elongated shaft having a first end and a distal second end;
pressing a blunt end member against a tissue of a patient, the blunt end member at the first end of the elongated shaft;
passing a needle of a syringe through a bore and into the tissue, the bore is in the blunt end member, the bore having an inner diameter greater than an outer diameter of the needle;
injecting a fluid from the syringe into the tissue by pressing a plunger of the syringe;
removing the needle from the tissue and from the bore; and
releasing the blunt end member from the tissue.

11. The method of claim 10, wherein the fluid is an anesthetic.

12. The method of claim 10, wherein the blunt end member has a curved surface for pressing against a tissue of a patient.

13. The device for reducing pain of claim 10, wherein the blunt end member is coated with a slip-reducing material.

14. The device for reducing pain of claim 4, wherein the slip-reducing material is rubber.

15. A device for reducing pain, the device comprising:
an elongated shaft having a first end and a distal second end; and
a blunt end member at the first end of the elongated shaft, the blunt end member having a rounded surface facing away from the elongated shaft with a bore passing through the blunt end member and the rounded surface, the bore having an inner diameter greater than an outer diameter of a needle.

16. The device for reducing pain of claim 19, wherein the curved surface is coated with a slip-reducing material.

17. The device for reducing pain of claim 15, further comprising a dental device affixed at the distal second end of the elongated shaft.

18. The device for reducing pain of claim 17, wherein the dental device is a dental mirror.

19. The device for reducing pain of claim 17, wherein the dental device is a dental pick.

20. The device for reducing pain of claim 17, wherein the dental device is a dental scraper.
